(19) United States
(12) Patent Application Publication
(10) Pub. No.: US 2007/0179299 A1
(43) Pub. Date: Aug. 2, 2007

(54) Dioxaborines as organic n-semiconductors, process for the production of semiconductors utilizing dioxaborines, and semiconductor component, field effect transistor, and diode having a dioxaborine

(75) Inventors: Marcus Halik, Erlangen (DE); Gunter Schmid, Hemhofen (DE); Lisa Davis, Nottingham (GB)

Correspondence Address: LERNER GREENBERG STEMER LLP, P O BOX 2480, HOLLYWOOD, FL, 33022-2480, US

(73) Assignee: Infineon Technologies AG

(21) Appl. No.: 10/993,117

(22) Filed: Nov. 19, 2004

Related U.S. Application Data
(62) Division of application No. 10/281,828, filed on Oct. 28, 2002, now abandoned.

(30) Foreign Application Priority Data
Oct. 26, 2001 (DE) 101 52 938.4

(51) Int. Cl. C07D 231/00

(52) U.S. Cl. 548/110

(57) ABSTRACT

Dioxaborines as organic n-semiconductors, a process for the production of semiconductors utilizing dioxaborines, and a semiconductor component, a field effect transistor, and a diode having a dioxaborine are provided. Dioxaborines have a conjugated π-system that carries two terminal six-membered dioxaborine heterocycles that are electronically linked to one another via the central π-system. The compounds have good electron mobility and very good reversibility of redox behavior and are therefore suitable as organic semiconductors in electronic semiconductor components. Processes for manufacturing the electronic semiconductor components utilize the dioxabroines.

### CROSS-REFERENCE TO RELATED APPLICATIONS

This is a divisional application of application Ser. No. 10/281,828, filed Oct. 28, 2002; the application also claims the priority, under 35 U.S.C. §119, of German patent application No. 101 52 938.4, filed Oct. 26, 2001; the prior applications are herewith incorporated by reference in their entirety.

### BACKGROUND OF THE INVENTION

### Field of the Invention

The invention relates to dioxaborines that have n-semiconductor properties, an electronic component that includes such dioxaborines, and a process for the production of such a semiconductor component.

Electronic semiconductor chips are widely used in a variety of technical applications. However, their production is still very complicated and expensive. Silicon substrates can be thinned to very small layer thicknesses so that they become flexible. However, these processes are very expensive, so that flexible or curved microchips are suitable only for demanding applications where high costs can be accepted. The use of organic semiconductors offers the possibility of economical production of microelectronic semiconductor circuits on flexible substrates. An example of an application is a thin film with integrated control elements for liquid crystal displays. A further application is transponder technology, where information about a product is stored on tags.

Organic semiconductors can be very simply structured, for example by printing processes. However, the use of such organic semiconductors is at present still limited by the low mobility of charge carriers in the organic polymeric semiconductors. This is currently not more than 1 to 2 cm²/Vs. The maximum operating frequency of transistors and hence of the electronic circuit is limited by the mobility of the charge carriers, holes, or electrons. Mobilities of the order of magnitude of 10⁻¹ cm²/Vs are sufficient for a driver application in the production of TFT active matrix displays. For high-frequency applications, however, the organic semiconductors are unsuitable to date. For technical reasons, wireless information transmission (RF-ID systems) are possible only above a certain minimum frequency. In systems that draw their energy directly from the alternating electromagnetic field and hence have no voltage supply of their own, carrier frequencies of 125 kHz or 13.56 MHz are widely used. Such systems are used, for example, for identifying or marking articles in smartcards, ident tags or electronic stamps.

In order to improve charge carrier transport in organic semiconductors, processes in which semiconducting molecules, for example pentacene or oligothiophenes, can be deposited as far as possible in an ordered manner have been developed. This is possible, for example, by vacuum sublimation. Ordered deposition of the organic semiconductor increases the crystallinity of the semiconductor material. As a result of the improved π-π overlap between the molecules or the side chains, the energy barrier for the charge carrier transport can be lowered. By substituting the semiconducting molecular units by bulky groups in the deposition of the organic semiconductor from the liquid or gas phase, it is possible to produce domains that have liquid crystalline properties. Furthermore, synthesis processes in which as high a regioregularity as possible is achieved in the polymers by the use of asymmetric monomers have been developed.

The above-described mobilities of 1 to 2 cm²/Vs of charge carriers in organic semiconductors have been measured to date almost exclusively in the case of organic materials that exhibit hole charge transport. This limits the use of organic materials to slow circuits having a high power consumption (pMOS circuits). In order to be able to produce fast circuits having a low power consumption (CMOS) or to construct organic diodes, however, materials having high electron mobility are also required in addition to materials having high hole mobility.

The organic materials known to date and having electron transport properties generally have low electron mobilities that moreover depend greatly on the ambient conditions and are sensitive, for example, to oxygen. These compounds are processed by vaporization techniques. In organic light emitting diodes, for example, compounds of the Alq₃ type (tris(8-hydroxyquinolinato)aluminum) are used. These compounds have mobilities of less than 10⁻⁶ cm²/Vs. Furthermore, compounds of the oxadiazole type [2-(4-biphenyl)-5-(4-tert-butylphenyl)-1,2,3-oxadiazole] have been used for the production of organic light emitting diodes. The charge carriers have mobilities of less than 10⁻⁶ cm²/Vs. Furthermore, H. E. Katz et al., Nature, 404, (2000), 478-81, describe organic semiconductor compounds of the naphthalenetetracarboxylic acid diimide type, which reach charge carrier mobilities of 0.1 cm²/Vs.

Dioxaborine compounds are used, for example, as emitter dyes in organic light emitting diodes. Such compounds are described, for example, in Japanese Patent Application Nos. JP 2000159777 and JP 11335368.

Furthermore, dioxaborines are used as sensitizers in photographic recording materials, in order to extend the photographic sensitivity of a silver halide-containing photographic film beyond the intrinsic sensitivity range. Such dioxaborines suitable as photographic sensitizers are described, for example, in German published, non-prosecuted patent application DE 19646111, and East German Patent Nos. DD 220728 and DD 286241. Furthermore, the use of dioxaborines as laser dyes is known. Suitable dioxaborines are described, for example, in East German Patent No. DD 225884, and U.S. Pat. Nos. 3,898,218, 3,959,480, and 3,936,488.

### SUMMARY OF THE INVENTION

It is accordingly an object of the invention to provide dioxaborines as organic n-semiconductors, a process for the production of semiconductors utilizing dioxaborines, and a semiconductor component, a field effect transistor, and a diode having a dioxaborine that overcome the hereinafore-mentioned disadvantages of the heretofore-known compounds, devices, and processes of this general type that have high electron mobility, are simple to prepare, and can be simply and economically processed. With the foregoing and other objects in view, there is provided, in accordance with the invention, a dioxaborines of the Formula I:

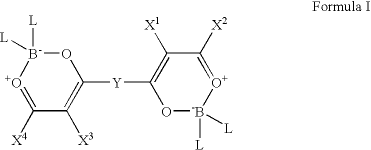

In Formula I, Y is a bivalent substituent that includes a conjugated π-electron system that extends between the six-membered dioxaborine ring bonded to the radical Y. X, in each case independently for each position, is a hydrogen atom, an alkyl group, a cycloalkyl group, or an aryl group, it being possible in these groups for some or all of the hydrogen atoms also to be replaced by fluorine atoms and for the aryl groups also to carry further substituents. L, in each case independently for each position, is a fluorine atom, a monodentate ligand, or a bidentate chelate ligand formed by two L bonded to a boron atom.

The dioxaborines of the Formula I have electron mobilities in the range from 10⁻³ to 10⁻¹ cm²/Vs, making these materials also suitable for realizing fast circuits having a low power consumption. The high electron mobility in the materials according to the invention is achieved by virtue of the fact that a π-conjugated system is substituted by dioxaborine heterocycles in a manner such that these electronically interact directly with the π system. For this purpose, group Y of the Formula I links the two terminal six-membered dioxaborine rings by a conjugated π-electron system. The group Y can therefore have considerable variety in its structure, but it must be ensured that the two dioxaborine heterocycles are linked to one another via a π-electron system.

The electronic properties of the compounds according to the invention can be modified by the substi,tuents X¹ to X⁴. X¹ and X³ are preferably a hydrogen atom, while the radicals X² and X⁴ are a hydrocarbon radical, which may also contain one or more heteroatoms, for example oxygen, nitrogen, or sulfur, it being possible for some or all of the hydrogen atoms of the hydrocarbon radical also to be replaced by fluorine atoms. The substituents X¹ to X⁴ may be identical or different. If one of the substituents X¹ to X⁴ is formed by an alkyl group, this preferably includes 1 to 10 carbon atoms,-it being possible for the alkyl group to be straight-chain or branched, i.e. for it also to contain one or more secondary or tertiary carbon atoms. The substituents X¹ to X⁴ may also be a cycloalkyl group. This preferably includes 5 to 10 carbon atoms and may include one or more hydrocarbon rings. A cyclohexyl group is particularly preferred. The substituents X¹ to X⁴ may also be an aryl group. This preferably includes 6 to 14 carbon atoms and may include one or more aromatic rings that may be fused or may be linked via a single bond or a bivalent alkyl group having 1 to 6 carbon atoms, and is preferably a phenyl group. The aryl groups may carry substituents, in particular alkyl groups having 1 to 10 carbon atoms or alkoxy groups having 1 to 10 carbon atoms. The alkyl and alkoxy groups may be straight-chain or branched. Two substituents X¹, X² or X³ and X⁴ together may also form a cyclic substituent, in particular a six-membered ring, to which in turn aromatic rings may be fused. These aromatic rings preferably being linked in such a way that the π-electron system of the dioxaborine is further delocalized. A combination of the abovementioned groups may form the individual substituents X¹ to X⁴.

The ligands L bonded to boron are preferably a fluorine atom. Acetyl groups as well as aryl groups are also suitable, these preferably having 6 to 14 carbon atoms. Furthermore, two ligands L bonded to a boron atom preferably form a bidentate chelate ligand, the coordination sites of the chelate ligand preferably being formed by oxygen.

The group Y, which provides a π-conjugated link between the two dioxaborine heterocycles, may have a very wide variety of structures. Y is preferably selected from the group including bivalent aryl groups, bivalent heteroaryl groups, bivalent polyenes, bivalent ethynylenes, and combinations of the groups. The bivalent aryl groups preferably include 6 to 20 carbon atoms, it being possible for these groups also to carry further substituents, in particular alkyl groups having 1 to 10 carbon atoms, and it being possible for the alkyl groups to be straight-chain or branched. The bivalent heteroaryl groups preferably contain oxygen, nitrogen or sulfur as a heteroatom, it being possible for the heteroaryl group also to contain a plurality of heteroatoms that are identical or different. The bivalent heteroaryl groups preferably contain 4 to 20 carbon atoms and 1 to 5 heteroatoms, which may be identical or different. The bivalent polyenes and the bivalent ethynylenes preferably include 2 to 20 carbon atoms, it being possible for the polyenes also to be mono- or polysubstituted, in particular by halogen atoms, hydrocarbon radicals, and heteroaryl radicals, which may also be further substituted. The polyene system may also include one or more hydrocarbon rings. These groups may be combined with one another to give extensive π-electron systems which range between the two terminal dioxaborine heterocycles which form the terminal groups.

If Y includes an aryl group, this is preferably selected from the following group:

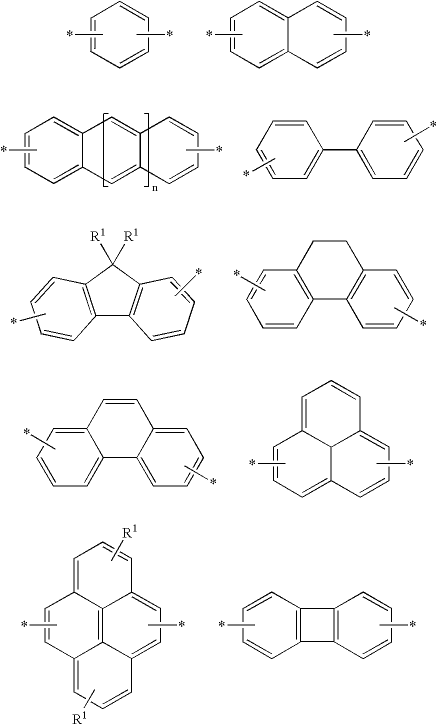

In the preceding formula, R¹, in each case independently for each position, is a hydrogen atom, an alkyl group that preferably includes 1 to 10 carbon atoms, a cycloalkyl group that preferably includes 5 to 20 carbon atoms, an alkoxy group having preferably 1 to 10 carbon atoms, an aryl group that preferably includes 6 to 20 carbon atoms, or an aryloxy group that preferably includes 6 to 20 carbon atoms. It also is possible for these groups for some or all of the hydrogen atoms to be replaced by fluorine atoms. In addition, n is an integer between 1 and 3. If Y includes at least one heteroaryl group, this is preferably selected from the following group:

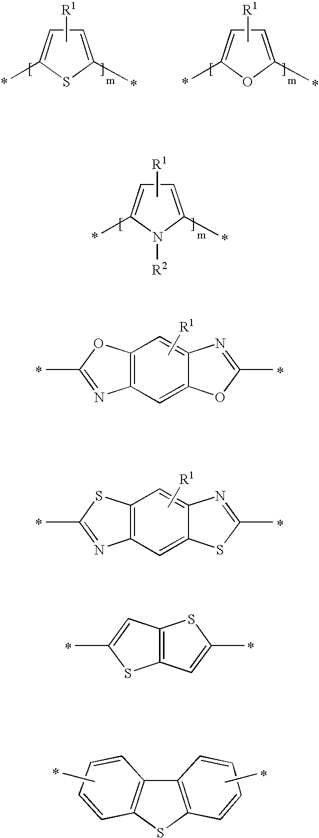

In these formulas, R¹, in each case independently for each position, may have the abovementioned meaning. R² is a hydrogen atom, an alkyl group that preferably includes 1 to 10 carbon atoms, a cycloalkyl group that preferably includes 5 to 20 carbon atoms, an alkoxy group having preferably 1 to 10 carbon atoms, an aryl group or an aryloxy group, the last-mentioned groups preferably including 6 to 20 carbon atoms. In said groups, some or all of the hydrogen atoms may also be replaced by fluorine atoms. Furthermore, m is an integer between 1 and 6. If Y includes at least one polyene and/or one ethynylene group, this is preferably selected from the following group:

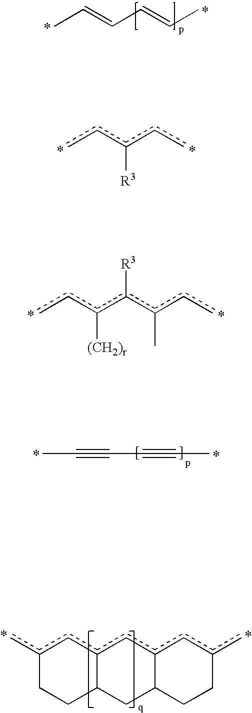

In the preceding formulas, R³ is a hydrogen atom, a halogen atom, in particular a chlorine atom, an alkyl group that preferably includes 1 to 10 carbon atoms, or an aryl group that preferably includes 6 to 20 carbon atoms, or R³ is selected from the following group:

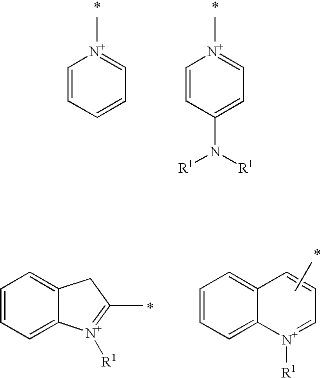

In the preceding formula, R¹ has the abovementioned meaning. p is an integer between 0 and 5. q is 0 or 1. r is 1 or 2.

The groups described above may be combined with one another as desired to give extensive π-systems that form the bivalent group Y. Examples of possible combinations are shown below.

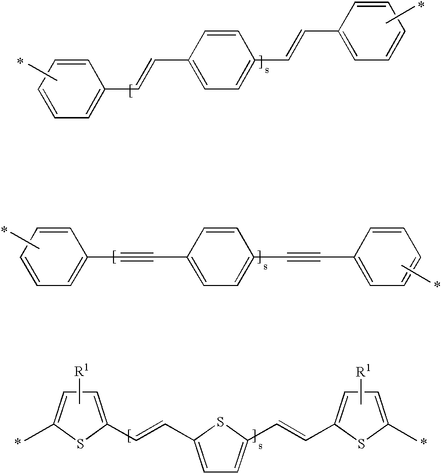

In these formulas, R¹ has the abovementioned meaning and s is an integer between 0 and 3.

In a preferred group of the dioxaborines, in each case, two of the ligands L bonded to boron form a bidentate chelate ligand, the ligand preferably being bonded to boron by an oxygen.

In this case, the chelate ligand is preferably selected from the following group:

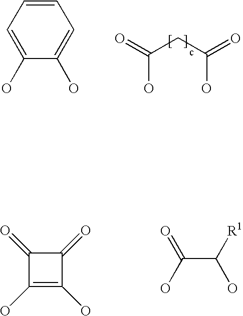

In these formulas, R¹ has the abovementioned meaning and t is an integer between 0 and 2.

The compounds described above are readily obtainable by customary synthesis processes. They have low reduction potentials and hence, when used as organic semiconductors, a low barrier for the injection of charge carriers. Furthermore, the compounds exhibit very good reversibility in redox behavior. The compounds are therefore very suitable for use in organic semiconductor components.

With the objects of the invention in view, there is also provided a semiconductor component that includes one or more of the dioxaborines described above. The semiconductor element is particularly preferably a field effect transistor or an organic diode.

With the objects of the invention in view, there is also provided processes for the production of semiconductor components. The processes utilize the quality of the dioxaborines, specifically, that they can be readily processed and are thermally stable to such an extent that they can be vaporized. In the first step of the process, a substrate is provided, and a layer of a dioxaborine as described above is applied to the substrate and electrical contacts being made with the layer of the dioxaborines. The exact process sequence is determined by the structure of the desired semiconductor component. Thus, in the production of an organic field effect transistor, for example, the metallic contacts serving as source electrode, drain electrode and gate electrode can first be deposited on a flexible substrate, for example a polymer film, the gate electrode can then be insulated with a dielectric and then a layer of the dioxaborine can be applied as an organic semiconductor. The structure of such a transistor and hence the sequence in its production can be varied in the customary manner known to a person skilled in the art. Thus, it is also possible, for example, first to deposit a gate electrode and to insulate it with a gate dielectric, in order then to apply a layer of dioxaborines as an organic semiconductor on said dielectric and finally to deposit the contacts for the source electrode and drain electrode on the layer of the dioxaborine.

For the deposition on the substrate, the dioxaborines can first be dissolved in a solvent. The solution then can be applied to the substrate by spin-coating or printing. According to a further process variant, the dioxaborines can also be applied to the substrate by vapor deposition.

Other features that are considered as characteristic for the invention are set forth in the appended claims.

Although the invention is illustrated and described herein as embodied in dioxaborines as organic n-semiconductors, a process for the production of semiconductors utilizing dioxaborines, and a semiconductor component, a field effect transistor, and a diode having a dioxaborine, it is nevertheless not intended to be limited to the details shown, because various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying examples.

### DESCRIPTION OF THE PREFERRED EMBODIMENTS

Where the dioxaborines described in examples 1 to 13 are purified by sublimation, the sublimation is suitably carried out at pressures of 10⁻⁶ to 10⁻⁷ mmHg and temperatures above 100° C.

### EXAMPLE 1

### Dioxaborine 1

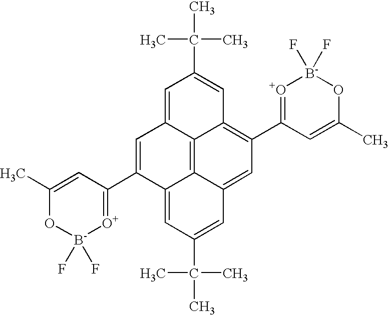

A solution of 3.98 g (10 mmol) of 4,9-diacetyl-2,7-di-tert-butylpyrene ⁽¹⁾ in 8.16 g (80 mmol) of acetic anhydride is added dropwise, at a temperature of 60° C. over 3 hours, to a mixture of 7.52 g (40 mmol) of a boron trifluoride/acetic acid complex and 8.16 g (80 mmol) of acetic anhydride while stirring. The reaction mixture is stirred for a further 8 hours at 60° C. and, after cooling to room temperature, the precipitate is filtered off with suction and washed with a little ethyl acetate and diethyl ether.

For the purification, the solid is recrystallized from acetic anhydride and chromatographed over silica gel (solvent -dichloromethane). The yield is 3.6 g (62%) of yellow powder having a melting point 316° C. Further purification is carried out by sublimation.

### EXAMPLE 2

### Dioxaborine 2

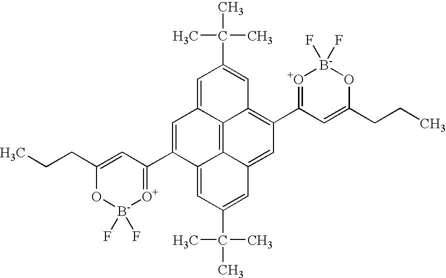

A solution of 3.98 g (10 mmol) of 4,9-diacetyl-2,7-di-tert-butylpyrene ⁽¹⁾ in 12.65 g (80 mmol) of butyric anhydride is added dropwise, at a temperature of 60° C. over 3 hours, to a mixture of 7.52 g (40 mmol) of a boron trifluoride/acetic acid complex and 12.65 g (80 mmol) of butyric anhydride while stirring. The reaction mixture is stirred for a further 8 hours at 60° C. After cooling to room temperature, the precipitate is filtered off with suction and washed with a little ethyl acetate and diethyl ether.

For the purification, the solid is recrystallized from acetic anhydride and chromatographed over silica gel (solvent—dichloromethane). The yield is 4.5 g (71%) of yellow powder having a melting point above 300° C. Further purification is carried out by sublimation.

### EXAMPLE 3

### Dioxaborine 3

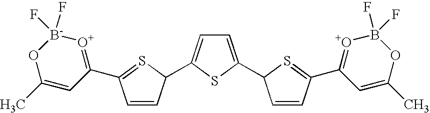

A solution of 1 g (3 mmol) of 2,5′-diacetyl-tert-thiophene in 2.45 g (24 mmol) of acetic anhydride is added dropwise, at a temperature of 60° C. over 3 hours, to a mixture of 2.26 g (12 mmol) of a boron trifluoride/acetic acid complex and 2.45 g (24 mmol) of acetic anhydride while stirring. The reaction mixture is stirred for a further 8 hours at 60° C. and, after cooling to room temperature, the precipitate is filtered off with suction and washed with a little ethyl acetate and diethyl ether.

For the purification, the solid is recrystallized from acetic anhydride and chromatographed over silica gel (solvent—dichloromethane). The yield is 1.1 g (72%) of yellow powder—melting point 245° C. Further purification is carried out by sublimation.

### EXAMPLE 4

### Dioxaborine 4

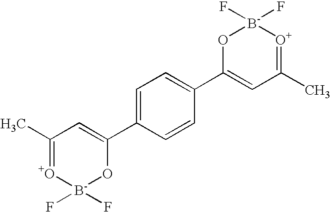

A solution of 1.62 g (10 mmol) of 1,4-diacetylbenzene in 2.45 g (24 mmol) of acetic anhydride is added dropwise, at a temperature of 60° C. over 3 hours, to a mixture of 7.52 g (40 mmol) of a boron trifluoride/acetic acid complex and 8.15 g (80 mmol) of acetic anhydride while stirring. The reaction mixture is stirred for a further 8 hours at 60° C. After cooling to room temperature, the precipitate is filtered off with suction and washed with a little ethyl acetate and diethyl ether.

For the purification, the solid is recrystallized from acetic anhydride and chromatographed over silica gel (solvent—dichloromethane). The yield is 86% of a pale yellow powder—melting point 295-7° C. Further purification is carried out by sublimation.

### EXAMPLE 5

### Dioxaborine 5

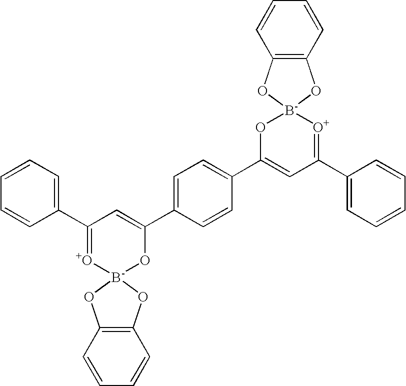

A mixture of 3.74 g (10 mmol) of dicarbonyl compound 1⁽²⁾, prepared from a diacetyl compound and the corresponding ethyl benzoate through ester condensation according to Organikum, Deutscher Verlag der Wissenschaften, Berlin (1999), 1.24 g (20 mmol) of boric acid and 2.2 g (20 mmol) of pyrocatechol, in 500 ml of 1,2-dichloroethane, is refluxed for 12 hours. After cooling to room temperature, the precipitate is filtered off with suction and washed with a little ethyl acetate and diethyl ether.

For the purification, the solid is chromatographed over silica gel (solvent—dichloromethane). The yield is 76% of a yellow powder having a melting point above 300° C. Further purification is carried out by sublimation.

### EXAMPLE 6

### Dioxaborine 6

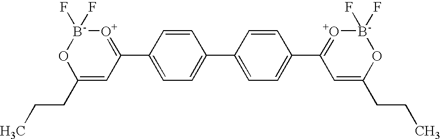

A solution of 2.38 g (10 mmol) of 4,4-diacetylbiphenyl in 12.65 g (80 mmol) of butyric anhydride is added dropwise, at a temperature of 60° C. over 3 hours, to a mixture of 7.52 g (40 mmol) of a boron trifluoride/acetic acid complex and 12.65 g (80 mmol) of butyric anhydride. The reaction mixture is stirred for a further 8 hours at 60° C. After cooling to room temperature, the precipitate is filtered off with suction and washed with a little ethyl acetate and diethyl ether.

For the purification, the solid is recrystallized from acetic anhydride and chromatographed over silica gel (solvent—dichloromethane). The yield is 54% of a pale yellow powder having a melting point >300° C. Further purification is carried out by sublimation.

### EXAMPLE 7

### Dioxaborine 7

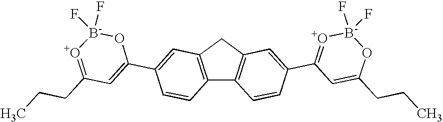

A solution of 2.50 g (10 mmol) of 2,7-diacetylfluorene in 12.65 g (80 mmol) of butyric anhydride is added dropwise, at a temperature of 60° C. over 3 hours, to a mixture of,7.52 g (40 mmol) of a boron trifluoride/acetic acid complex and 12.65 g (80 mmol) of butyric anhydride while stirring. The reaction mixture is stirred for a further 8 hours at 60° C. After cooling to room temperature, the precipitate is filtered off with suction and washed with a little ethyl acetate and diethyl ether.

For the purification, the solid is recrystallized from acetic anhydride and chromatographed over silica gel (solvent—dichloromethane). The yield is 66% of a yellow powder having a melting point >300° C. Further purification is carried out by sublimation.

### EXAMPLE 8

### Dioxaborine 8

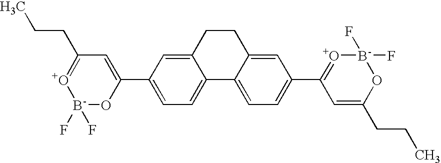

A solution of 2.64 g (10 mmol) of 2,7-diacetyl-9,10-dihydronaphthene in 12.65 g (80 mmol) of butyric anhydride is added dropwise, at a temperature of 60° C. over 3 hours, to a mixture of 7.52 g (40 mmol) of a boron trifluoride/acetic acid complex and 12.65 g (80 mmol) of butyric anhydride while stirring. The reaction mixture is stirred for a further 8 hours at 60° C. After cooling to room temperature, the precipitate is filtered off with suction and washed with a little ethyl acetate and diethyl ether.

For the purification, the solid is recrystallized from acetic anhydride and chromatographed over silica gel (solvent—dichloromethane). The yield is 39% of a yellow powder having a melting point >300° C. Further purification is carried out by sublimation.

### EXAMPLE 9

### Dioxaborine 9

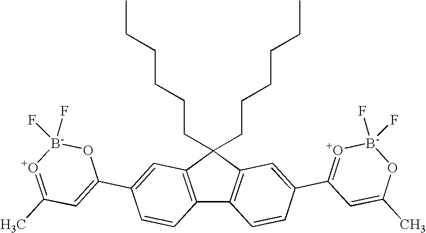

A solution of 4.18 g (10 mmol) of 2,7-diacetyl-9,9′-dihexylfluorene in 8.16 g (80 mmol) of acetic anhydride is added dropwise, at a temperature of 60° C. over 3 hours, to a mixture of 7.52 g (40 mmol) of a boron trifluoride/acetic acid complex and 8.16 g (80 mmol) of acetic anhydride while stirring. The mixture is evaporated to dryness in a rotary evaporator and then chromatographed over silica gel (solvent—dichloromethane). The yield is 38% of a yellow glassy solid. Further purification is carried out by sublimation.

### EXAMPLE 10

### Dioxaborine 10

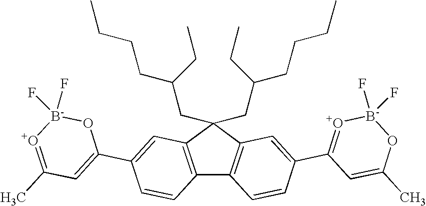

A solution of 4.74 g (10 mmol) of 2,7-diacetyl-9,9′-diisooctylfluorene in 8.16 g (80 mmol) of acetic anhydride is added dropwise, at a temperature of 60° C. over 3 hours, to a mixture of 7.52 g (40 mmol) of a boron trifluoride/acetic acid complex and 8.16 g (80 mmol) of acetic anhydride while stirring. The reaction mixture is stirred for a further 8 hours at 60° C. and is cooled, and then 20 g of silica gel are added. The mixture is evaporated to dryness in a rotary evaporator and then chromatographed over silica gel (solvent—dichloromethane). The yield is 42% of a yellow, highly—viscous oil.

### EXAMPLE 11

### Dioxaborine 11

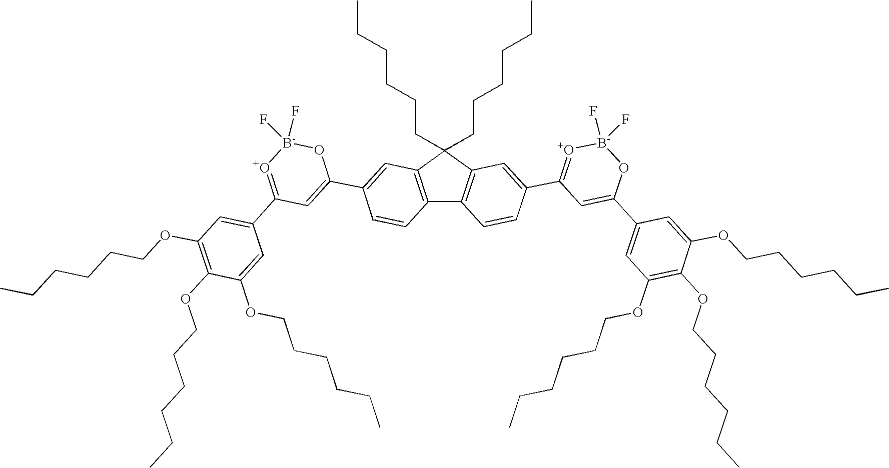

0.92 g (4.9 mmol) of a boron trifluoride/acetic acid complex is added dropwise to a solution of 3 g (2.4 mmol) of dicarbonyl compound 2⁽²⁾, prepared from a diacetyl compound and a corresponding ethyl benzoate by using ester condensation according to Organikum, loc. cit., in 100 ml of acetic acid, and the mixture is refluxed for 5 minutes. After cooling, 20 g of silica gel are added and the mixture is evaporated to dryness in a rotary evaporator. The mixture is chromatographed over silica gel (solvent—dichloromethane). The yield is 88% of a yellow highly viscous oil.

### EXAMPLE 11a

### Dioxaborine 11a

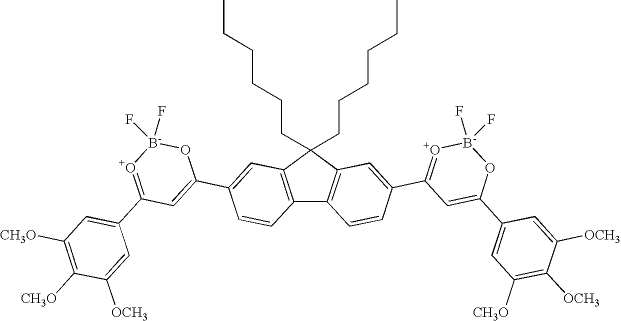

0.92 g (4.9 mmol) of a boron trifluoride/acetic acid complex is added dropwise to a solution of 1.94 g (2.4 mmol) of dicarbonyl compound 2⁽²⁾, prepared from a diacetyl compound and a corresponding ethyl benzoate by using ester condensation according to Organikum, loc. cit., in 100 ml of acetic acid, and the mixture is refluxed for 5 minutes. After cooling, 20 g of silica gel are added and the mixture is evaporated to dryness in a rotary evaporator. Chromatography over silica gel (solvent—dichloromethane) is then effected. The yield is 92% of a yellow highly viscous oil.

### EXAMPLE 12

### Dioxaborine 12

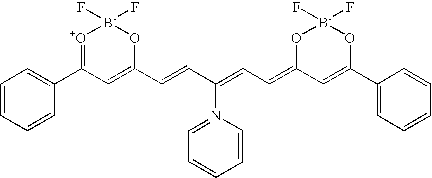

Two grams (2 g=20 mmol) of triethylamine are added dropwise, at a temperature of 70° C., to a mixture of 2 g (9.5 mmol) of 6-methyl -4-phenyl-2,2-difluoro-1,3,2-(2H)-dioxaborine ⁽³⁾ and 1.61 g (4 mmol) of cyanine former 1⁽⁴⁾ in 80 ml of acetonitrile and 10 ml of acetic anhydride. Stirring is carried out for a further 10 minutes and, after cooling, the solid is filtered off with suction. For the purification, the solid is recrystallized from acetic anhydride and chromatographed over silica gel (solvent—dichloromethane). The yield is 78% of a blue-gray powder having a melting point of 280° C. Further purification is carried out by sublimation.

### EXAMPLE 13

### Dioxaborine 13

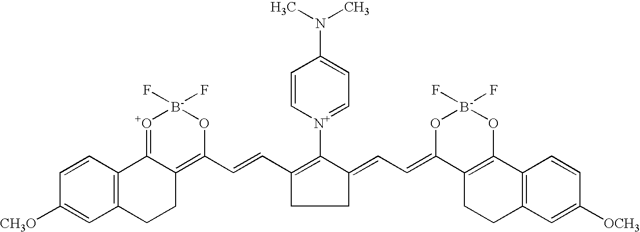

Two grams (2 g=20 mmol) of triethylamine are added dropwise, at a temperature of 70° C., to a mixture of 2.53 g (9.5 mmol) of methoxytetralone-dioxaborine ⁽⁵⁾ and 1.87 g (4 mmol) of cyanine former 2 ⁽⁶⁾ in 80 ml of acetonitrile and 10 ml of acetic anhydride. Stirring is carried out for a further 10 minutes and, after cooling, the solid is filtered off with suction.

For the purification, the solid is recrystallized from acetic anhydride and chromatographed over silica gel (solvent—dichloromethane). The yield is 78% of a golden powder—melting point 287° C. Further purification is carried out by sublimation.

### EXAMPLE 14

### Preparation of a Substrate Solution

Suitable solvents for the layer preparation are in principle all organic solvents whose boiling point is lower than the decomposition temperature of the dioxaborines and in which the compounds have a solubility of at least 0.1 percent by mass, e.g. chloroform, dichloromethane, THF, acetone, cyclohexanone, ethyl acetate, toluene, cresol, γ-butyrolactone, N-methylpyrrolidone and dimethylformamide.

In each case, 100 mg of the dioxaborines described under examples 1-3 are dissolved in 10 g of chloroform by shaking the mixture of the two components in a closed sample tube on a shaker for 1 hour. The solution is then filtered by using pressure filtration (filter size 0.2 μm) into a steam-cleaned sample tube in order to remove particles.

### EXAMPLE 15

### Film Preparation (Spin-Coating Technique)

A solution prepared as under example 14 is applied by spin-coating (1 000-5 000 rpm, 20 s, nitrogen atmosphere) to a suitable substrate on which transistor and/or circuit structures had been defined beforehand (e.g. Si wafer, glass or flexible sheet). The substrate is then dried for 2 minutes at 80° C. under an inert gas.

### EXAMPLE 16

### Film Preparation (Vapor Deposition)

A compound (i.e. examples 1-13) is applied to a substrate, as under example 15, by vapor deposition by using an evaporator. The evaporation times here depend on the desired layer thickness.

### EXAMPLE 17

### Film Preparation (Printing)

A solution prepared under example 14 is printed onto a suitable substrate by using a suitable template in a screen printing machine and then dried at 80° C.

### EXAMPLE 18

### Measurement of the Charge Carrier Mobilities

A field effect transistor processed as under examples 15-17 and include a gate electrode, a gate dielectric, and aluminum source and drain contacts is contacted by using a metal tip under an inert gas atmosphere on an analytical sampler. Using an electrical parameter measuring apparatus (for example one sold under the trademark AGILENT 4156), a transistor characteristic is measured. The charge carrier mobility is calculated from the characteristics. For compounds for examples 1-13, electron mobilities between 10⁻³ and 10⁻¹ cm²/Vs were determined in this manner.

### REFERENCES

⁽¹⁾ T. Yamato et al., Chem. Ber., 126 (1993), 2505-11;
⁽²⁾ Prepared from 1,4-diacetylbenzene by using ester condensation.
⁽³⁾ G. Goerlitz et al., Heteroatom. Chem., 8, (1997), 147.
⁽⁴⁾ M. Halik Thesis, http://sundoc.bibliothek.uni-halle.de/diss.online /99H017/index.htm.
⁽⁵⁾ D. Kaminski U.S. Pat. Nos. 3,898,218; 3,959,480; 3,936,488.
⁽⁶⁾ M. Halik et al., Chem. Eur. J., 5, 1999, 2511-2517.

1-15. (canceled)

16. A field effect transistor, comprising a substrate and a layer thereupon including a source electrode, a drain electrode, a gate electrode, and a dioxaborine having a formula 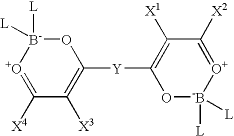
wherein
Y is a bivalent substituent having a conjugated π-electron system extending between six-membered dioxaborine heterocycles bonded thereto and including at least one member selected from the group consisting of bivalent aryl groups, bivalent heteroaryl groups, bivalent polyenes, bivalent ethynylenes, bivalent cyanines;
X¹, X², X³, X⁴ is a substituent independently selected from the group consisting of a hydrogen atom, an alkyl group, a cycloalkyl group, and an aryl group; and
L is a substituent independently selected from the group consisting of a fluorine atom, a monodentate ligand, and a bidentate chelate ligand bonded twice to said boron atom.

17-22. (canceled)

23. The field effect transistor according to claim 16, wherein at least one hydrogen in at least one of X¹, X², X³, X⁴ is replaced by a fluorine atom.

24. The field effect transistor according to claim 16, wherein at least one of X¹, X², X³, X⁴ is an aryl group carrying a further substituent.

25. The field effect transistor according to claim 16, wherein said Y aryl group is selected from the group of substituents consisting of: 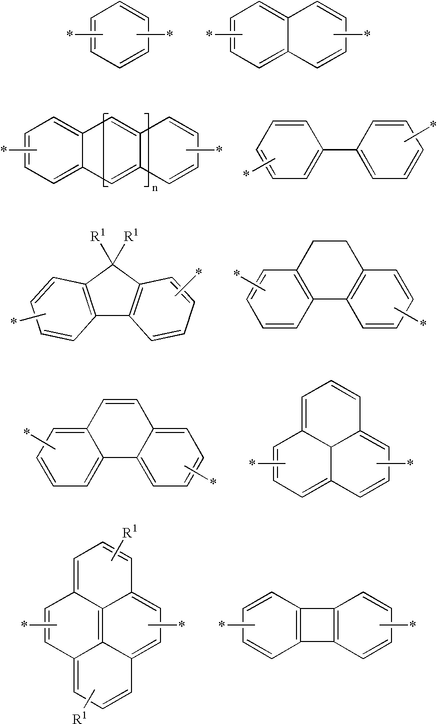
wherein R¹ is independently selected from the group consisting of a hydrogen atom, an alkyl group, a cycloalkyl group, an alkoxy group, an aryl group, and an aryloxy group; and n is an integer from 1 to 3.

26. The field effect transistor according to claim 25, wherein said R¹ has a hydrogen atom replaced by a fluorine atom.

27. The field effect transistor according to claim 16, wherein said Y heteroaryl group is selected from the group consisting of: 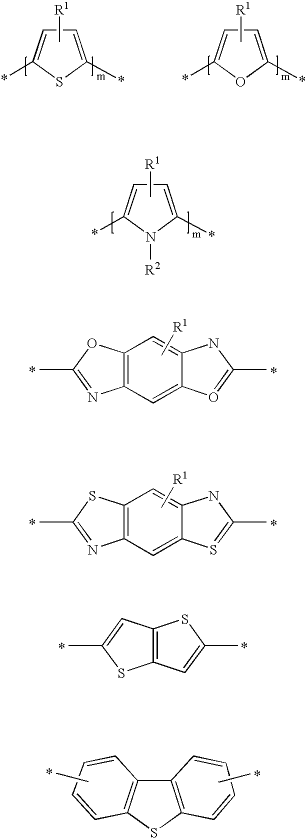
wherein R¹ is in each case independently selected from the group consisting of a hydrogen atom, an alkyl group, a cycloalkyl group, an alkoxy group, an aryl group, and an aryloxy group; and
R² is a substituent selected from the group consisting of a hydrogen atom, an alkyl group, a cycloalkyl group, an alkoxy group, an aryl group, and an aryloxy group; and
m is an integer from 1 to 6.

28. The field effect transistor according to claim 27, wherein R¹ has a hydrogen atom replaced by a fluorine atom.

29. The field effect transistor according to claim 27, wherein R² has a hydrogen atom replaced by a fluorine atom.

30. The field effect transistor according to claim 27, wherein said R² has each hydrogen substituted by a fluorine atom.

31. The field effect transistor according to claim 16, wherein said Y polyene and ethynylene groups have a formula selected from the group consisting of: 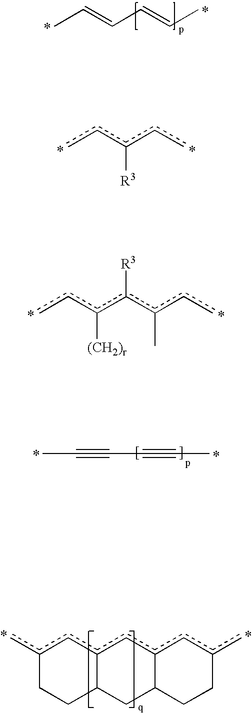
wherein:
R³ is selected from the group consisting of a hydrogen atom, a halogen atom, an alkyl group, an aryl group, 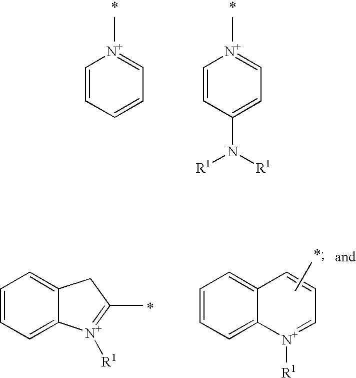
wherein R¹ is independently selected from the group consisting of a hydrogen atom, an alkyl group, a cycloalkyl group, an alkoxy group, an aryl group, and an aryloxy group;
p is an integer from 0 to 5;
q is an integer from 0 to 1; and
r is an integer from 1 to 2.

32. The field effect transistor according to claim 31, wherein R¹ has a hydrogen atom replaced by a fluorine atom.

33. The field effect transistor according to claim 16, wherein L is selected from the group consisting of: 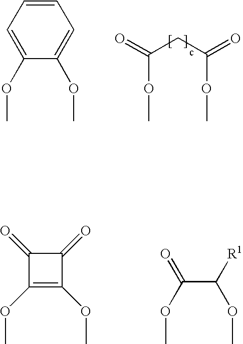
wherein
R¹ is independently selected from the group consisting of a hydrogen atom, an alkyl group, a cycloalkyl group, an alkoxy group, an aryl group, and an aryloxy group; and
t is an integer from 0 to 2.

34. The field effect transistor according to claim 33, wherein R¹ has a hydrogen atom replaced by a fluorine atom.

35. The field effect transistor according to claim 16, further comprising a gate electrode, a gate dielectric, and aluminum source and drain contacts.

36. The field effect transistor according to claim 16, having an electron mobility in the range from 10⁻³ to 10⁻¹ cm²/Vs.
